(19) United States
(12) Patent Application Publication
(10) Pub. No.: US 2009/0137876 A1
(43) Pub. Date: May 28, 2009

(54) DIAGNOSTIC CAPSULE WITH PERIPHERAL IMAGING

(75) Inventor: Christopher Philip BROPHY, Pittsford, NY (US)

Correspondence Address: HESLIN ROTHENBERG FARLEY & MESITI PC, 5 COLUMBIA CIRCLE, ALBANY, NY, 12203, US

(73) Assignee: BIOSCOPIX, INC., Canandaigua, NY (US)

(21) Appl. No.: 12/254,538

(22) Filed: Oct. 20, 2008

Related U.S. Application Data
(60) Provisional application No. 60/981,297, filed on Oct. 19, 2007.

(51) Int. Cl. A61B 1/06

(52) U.S. Cl. 600/167

(57) ABSTRACT

A diagnostic capsule is disclosed having at least one light source; a rotatable substrate having at least one optical bench; at least one photo-sensitive region; and a circumferential window having an axis substantially parallel to an axis of rotation of the rotatable substrate. Related systems for use with a diagnostic capsule are also disclosed. An endoscopic diagnostic map is further disclosed. The endoscopic diagnostic map has a first dimension corresponding to a rotational position of at least one optical bench on a rotatable substrate of a diagnostic capsule. The endoscopic diagnostic map also has a second dimension corresponding a traveled position of the diagnostic capsule. Related methods of generating an endoscopic

### RELATED APPLICATIONS

This application claims priority to U.S. provisional patent application 60/981,297 filed on Oct. 19, 2007 and entitled “DIAGNOSTIC CAPSULE WITH PERIPHERAL IMAGING.” This application hereby incorporates the 60/981,297 provisional application by reference in its entirety.

### FIELD

The claimed invention generally relates to compact diagnostic capsules, and more particularly to methods, apparati, and systems which can be used to collect scanning spot sensor data for analysis and/or the generation of an endoscopic diagnostic map.

### BACKGROUND

Although great strides in cancer treatment have been developed over the years, cancer still remains among the leading causes of death in humans. One of the driving factors in our ability to successfully fight cancer is the ability to detect cancerous tissue at an early stage. Early detection requires regular check-ups and is also dependent on the ability of physicians to inspect a variety of areas on and within a patient's body, depending on the type of cancer being screened for. While blood tests can be indicative of a cancerous condition within a person's body, they do not always determine the type of cancer and can not pin-point the exact location of the cancer. Therefore, a visual and/or imaging inspection is often more desirable, either on its own or in conjunction with other types of tests.

Visual and/or imaging inspections of portions of the gastro-intestinal (GI) tract have been made possible in the last century, for the determination of cancerous and other medical conditions, by using endoscope technology. An endoscope is a probe which is inserted either in the mouth or nose end of the alimentary canal or the anal end of the alimentary canal. A modern endoscope is fitted with an illumination source and a video camera or image sensor on its tip which can relay images of the areas it is manipulated into by a medical professional. The endoscopic probe is connected to an external monitor and/or image storage device by a cable. The probes are also manipulated and guided into place by an operator using the same or a different cable. While valuable, these types of endoscopic procedures risk tissue perforation and are uncomfortable for patients, often requiring the use of sedatives. Furthermore, there are still areas of the alimentary canal which can not be reached readily by an endoscope, simply because it is too difficult to manipulate the probe into certain highly twisted areas, such as the small intestine.

More recently, advances in micro-assembly and integration have made it possible to create endoscopic capsules which are small enough to be swallowed by a patient and which have no wires or cables connecting them the outside world. These endoscopic capsules wirelessly transmit image data to a receiver located outside of a patient's body as the endoscopic pill passes through the patient's body. An example of such a commercially available imaging capsule is the “Bio-Pill” from Given Imaging of Israel. A schematic illustration of a cross-section of the “Bio-Pill” endoscopic capsule 20 is shown in FIG. 1A. For the sake of illustration, the “Bio-Pill” 20 is shown as passing through a portion of the alimentary canal 22. The Bio-Pill 20 has a case 24 with a clear dome 26 on one end. Illumination light emitting diodes (LED's) 28 are arranged around an imaging lens 30 and oriented to emit light through the end dome 26. Reflected light is directed by the lens 30 onto an image sensor 32. The Bio-Pill 20 offers a claimed 140 degree viewing angle. The Bio-Pill 20 has room for batteries 34, and a transmitter 36 for relaying image information outside the Bio-Pill 20. Unfortunately, while the Bio-Pill and other existing endoscopic capsules have a relatively wide field of view, a large portion of the center of the field of view is the opening of the alimentary canal itself, which is devoid of tissue. FIG. 1B schematically illustrates a typical image of the alimentary canal which can be collected by existing endoscopic capsules like the Bio-Pill 20. Unfortunately, the wide angle regions of tissue 38 being imaged exist over a rapidly changing object distance leading to distortion and low scattered light collection.

Therefore, there is a need for a diagnostic capsule which is not limited to relatively low levels of scattered imaging light or field of view problems while still being able to differentiate abnormal tissue from normal tissue and image the alimentary canal at the same time. The diagnostic capsule will also preferably help reduce the amount of time patients need to spend in a medical facility and reduce the amount of medical professional time needed to assist with and analyze the data from the diagnostic capsule.

### SUMMARY

A diagnostic capsule is disclosed. The diagnostic capsule has at least one light source and a rotatable substrate having at least one optical bench. The diagnostic capsule also has at least one photo-sensitive region and a circumferential window having an axis substantially parallel to an axis of rotation of the rotatable substrate.

A diagnostic system is also disclosed. The diagnostic system comprises a diagnostic capsule. The diagnostic capsule has at least one light source and a rotatable substrate having at least one optical bench. The diagnostic capsule also has at least one photo-sensitive region and an at least partially transparent circumferential window having an axis substantially parallel to an axis of rotation of the rotatable substrate. The diagnostic capsule further has a transmitter configured to transmit sensor data from the at least one photo-sensitive region. The diagnostic system also comprises at least one receiver configured to receive the sensor data from the transmitter. The diagnostic system further comprises a receiver controller coupled to the at least one receiver, the receiver controller configured to store the sensor data.

A method of generating an endoscopic diagnostic map is disclosed. Spot sensor data are collected from at least one light sensor which is optically coupled to a focused illuminated area of tissue through a circumferential window in a diagnostic capsule by rotating at least one optical bench to scan the illuminated area of tissue. The collected spot sensor data are arranged in an array having a first dimension corresponding to a rotational position of the at least one optical bench and a second dimension corresponding to a traveled position of the diagnostic capsule in a direction substantially parallel to an axis of rotation of the optical bench. One or more of the collected spot sensor data are stored in the array at locations substantially corresponding to an associated rotational position of the at least one optical bench and an associated traveled position of the diagnostic capsule for each of the one or more spot sensor data.

An endoscopic diagnostic map is also disclosed. The endoscopic diagnostic map has a first dimension corresponding to a rotational position of at least one optical bench on a rotatable substrate of a diagnostic capsule. The endoscopic diagnostic map also has a second dimension corresponding a traveled position of the diagnostic capsule.

A further diagnostic capsule is disclosed. The diagnostic capsule has at least one illumination light source and at least one excitation light source. The diagnostic capsule also has a first annular detector region configured to sense light at a first wavelength, a second annular detector region configured to sense light at a second wavelength, and an annular imaging region configured to sense one or more visible wavelengths of light. The diagnostic capsule has a circumferential window having a window axis. The diagnostic capsule further has a rotatable substrate having a rotation axis which is substantially parallel to the window axis. The rotatable substrate also comprises an optical bench. The optical bench is configured to i) focus light from the illumination and excitation light sources on a spot outside the circumferential window; ii) focus reflected, backscattered, luminesced, or fluoresced light of the first wavelength from outside the circumferential window onto the first annular detector region; iii) focus reflected, backscattered, luminesced, or fluoresced light of the second wavelength from outside the circumferential window onto the second annular detector region; and iv) focus reflected, backscattered, luminesced, or fluoresced light of the one or more visible wavelengths from outside the circumferential window onto the annular imaging region. The diagnostic capsule further has a motor rotatably coupled to the rotatable substrate. The diagnostic capsule also has circuitry configured to transmit data based on the reflected, backscattered, luminesced, or fluoresced light focused on the first annual detector region, the second annular detector region, and the annular imaging region.

### BRIEF DESCRIPTION OF THE DRAWINGS

FIG. 1A schematically illustrates an example of a white light imaging capsule.

FIG. 1B schematically illustrates an image of the alimentary canal produced by the capsule of FIG. 1A.

FIG. 2 schematically illustrates a partially-exposed perspective view of a different embodiment of a diagnostic capsule.

FIGS. 3 and 4 schematically illustrate alternate embodiments of diagnostic systems utilizing an embodiment of a diagnostic capsule.

FIG. 5A schematically illustrates a cross-sectional view of another embodiment of a diagnostic capsule with selected details highlighted and rotated.

FIG. 5B schematically illustrates a cross-sectional view of the diagnostic capsule of FIG. 5A taken along sectional lines 5-5 while the diagnostic capsule is within an alimentary canal.

FIG. 6A schematically illustrates a cross-sectional view of another embodiment of a diagnostic capsule with selected details highlighted and rotated.

FIG. 6B schematically illustrates a cross-sectional view of the diagnostic capsule of FIG. 6A taken along sectional lines 6-6 while the diagnostic capsule is within an alimentary canal.

FIG. 7A schematically illustrates a cross-sectional view of another embodiment of a diagnostic capsule with selected details highlighted and rotated.

FIG. 7B schematically illustrates a cross-sectional view of the diagnostic capsule of FIG. 7A taken along sectional lines 7-7 while the diagnostic capsule is within an alimentary canal.

FIG. 8 is a partially exposed view of a portion of the alimentary canal schematically illustrating a spot scanning path of an embodiment of a diagnostic capsule as it passes through the alimentary canal.

FIG. 9 schematically illustrates an embodiment of a diagnostic capsule having an embodiment of a momentum compensator.

FIG. 10 illustrates an embodiment of a method of generating an endoscopic diagnostic map.

FIGS. 11 and 12 schematically illustrate embodiments of endoscopic diagnostic maps.

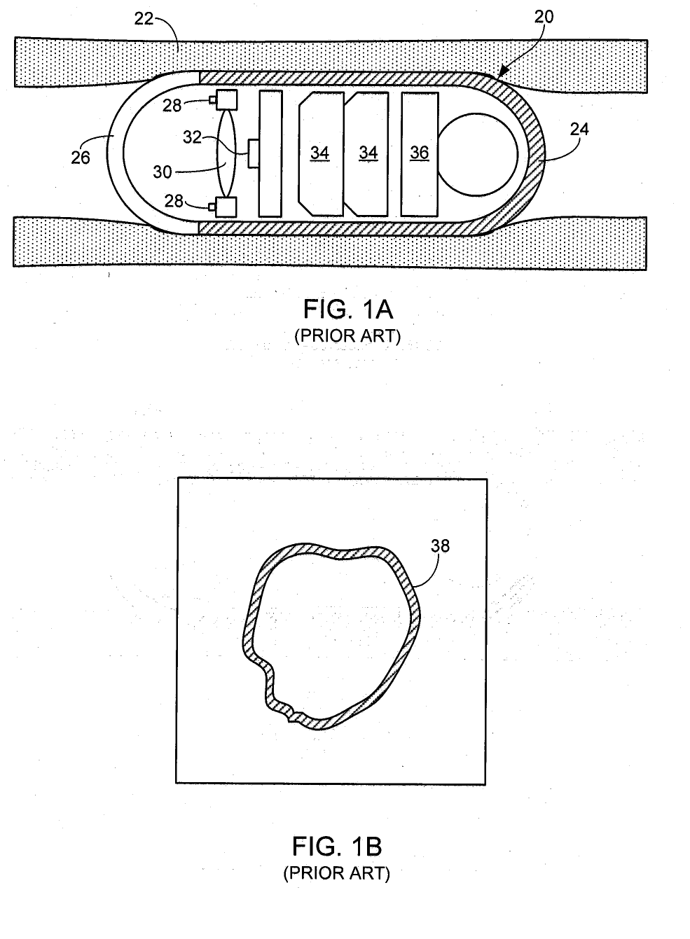

It will be appreciated that for purposes of clarity and where deemed appropriate, reference numerals have been repeated in the figures to indicate corresponding features, and that the various elements in the drawings have not necessarily been drawn to scale in order to better show the features.

### DETAILED DESCRIPTION

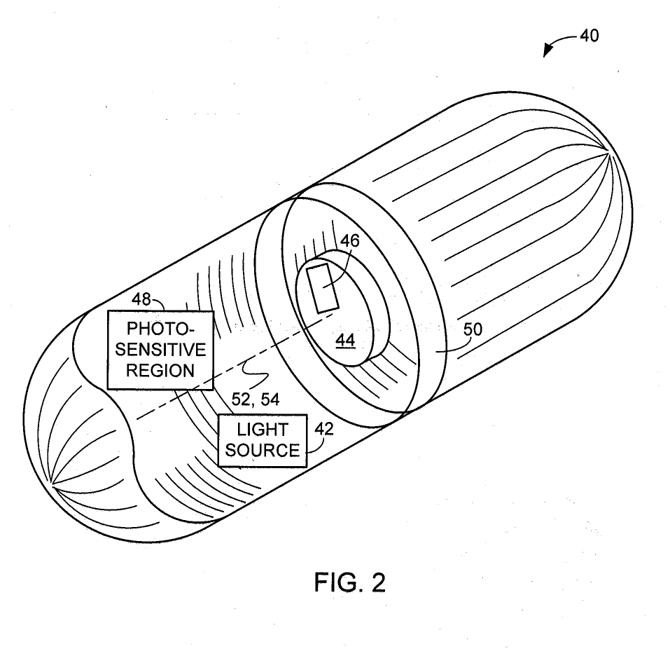

FIG. 2 schematically illustrates a partially-exposed perspective view of a different embodiment of a diagnostic capsule 40. The diagnostic capsule 40 has at least one light source 42. Suitable non-limiting examples of a light source 42 include a light emitting diode (LED) and a laser die. The diagnostic capsule 40 also has a rotatable substrate 44 having at least one optical bench 46. Various devices, not shown in this view, may be coupled to the rotatable substrate 44 to rotate the substrate 44, such as a micro-motor or a MEMS pancake motor. (“MEMS” stands for “micro electro-mechanical systems”). The diagnostic capsule 40 further has at least one photosensitive region 48. Examples of a suitable photo-sensitive region 48 include a detector sensitive to one or more wavelengths of light and an imaging sensor. In some embodiments, the photo-sensitive region 48 may also include light filtering elements to allow only desired wavelengths of light to pass on to the photo sensitive region 48. The diagnostic capsule 40 also has a circumferential window 50 having an axis 52 substantially parallel to an axis of rotation 54 of the rotatable substrate 44. In this embodiment, the two axes 52 and 54 are identical. The circumferential window 50 may be fully transparent or partially transparent, depending on the embodiment. The circumferential window 50 may be made of glass, quartz, a suitably transparent metal or alloy, plastic, or combinations thereof. The window 50 in this embodiment is illustrated as being preferably continuous around the circumference of the diagnostic capsule 40, however, in other embodiments, the window 50 does not need to be continuous.

The diagnostic capsule 40 may be sized to be ingestible by a mammal and travel down the alimentary canal in a direction substantially parallel to the circumferential window axis 52. Many portions of the alimentary canal are flexible and will tend to conform to the circumference of the diagnostic capsule 40, ensuring that for at least a portion of the diagnostic capsule's journey through the alimentary canal, the circumferential window 50 will be in contact with or generally facing the tissue of the alimentary canal. The optical bench 46 is configured in this embodiment to focus outgoing light from the at least one light source 42 through the circumferential window 50 and onto the tissue in contact with or facing the window 50. Since the optical bench 46 is coupled to or is an integral part of the rotatable substrate 44, as the substrate 44 rotates, a focused spot of outgoing light will be traced across the tissue around the diagnostic capsule 40 outside the window 50. The focused spot can be a variety of different sizes, depending on where the focal plane is chosen to be relative to the window 50. The smallest spot size will be dictated by the demagnification of the source image. The demagnification is greater as the lens element, along with a shorter focal length, moves closer to the circumferential window as shown in the figures. In some embodiments, for highest fluorescent radiance, the optical bench may be configured to focus on the tissue essentially in contact with the window, and the demagnification may be set by the position and focal length of the one or more lens elements. In some embodiments, the optical bench can be set to provide a defocused spot.

Thus, one or more wavelengths of light will be peripherally focused upon the tissue surrounding the capsule in a scanning fashion. Depending on the one or more wavelengths of incident light on the tissue and depending on the physiology of the tissue, the tissue may reflect, scatter, luminesce, or fluoresce light back through the circumferential window 50. In some embodiments, the physiology of the tissue may be enhanced with a contrast agent ingested by the subject prior to ingestion of the pill. Such a contrast agent may bond to certain types of tissue and fluoresce when excited by the diagnostic capsule or inherently luminesce. It should be understood, however, that many types of tissue do not need a contrast agent to exhibit useful fluorescence. The result is that one or more wavelengths of light may be scattered, reflected, or otherwise emitted back towards the capsule. The optical bench 46 is also configured to direct this incoming light which enters the circumferential window 50 onto the at least one photo-sensitive region 48. The diagnostic capsule 40 may also have circuitry (not illustrated) which is operatively coupled to the at least one photo-sensitive region 48 to capture spot light data at intervals from incoming light directed to the at least one photo-sensitive region 48 by the at least one optical bench 46. The capture intervals are akin to a sampling rate, and can be a fixed regular interval at all times, a varying regular interval, or irregular intervals. Shorter intervals correspond to higher sampling rates and more spot data points per inch.

The diagnostic capsule 40 may also have a transmitter (not shown) coupled to the circuitry. The circuitry may transmit the raw sensor data collected from the at least one photo-sensitive region 48, or it may process the data prior to transmission.

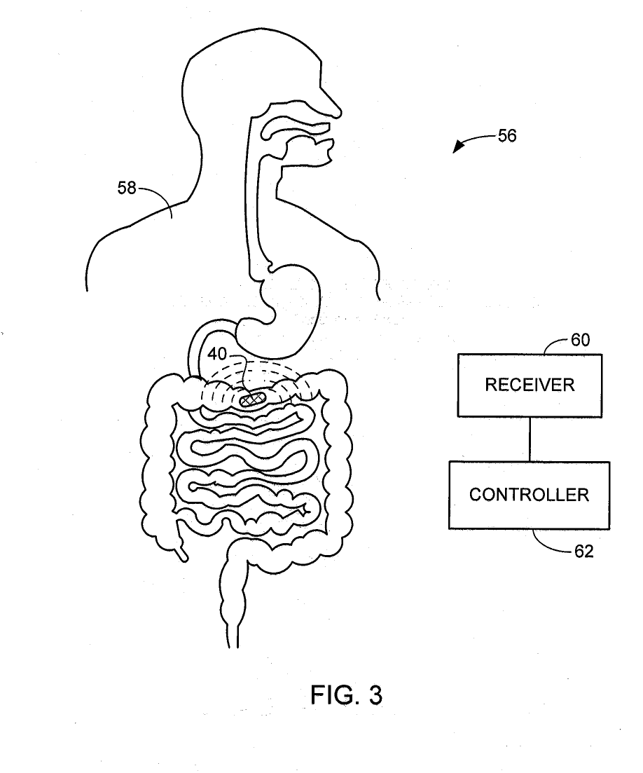

Since the embodied diagnostic capsules will be transmitting at least sensor data, they will preferably be used as part of a diagnostic system which is capable of receiving the transmitted data. FIG. 3 schematically illustrates one embodiment of a diagnostic system 56 which can be used in applications where a diagnostic capsule 40 is ingested by a subject 58 and moves through his/her alimentary canal. The diagnostic system 56 has a receiver 60 coupled to a receiver controller 62, both of which are located external to the subject 58. Since receiver 60 is configured to receive transmissions from diagnostic capsule 40, the receiver 60 should be positioned within transmission range of the diagnostic capsule 40. The receiver controller 62 may be configured to store the transmitted data received at the receiver 60 from the diagnostic capsule 40. The receiver 60, and optionally the receiver controller 62 may be portable and even wearable so that the subject 58 may carry the receiver 60 with him/her for increased freedom of movement during the relatively long time it can take a diagnostic capsule to move through the alimentary canal.

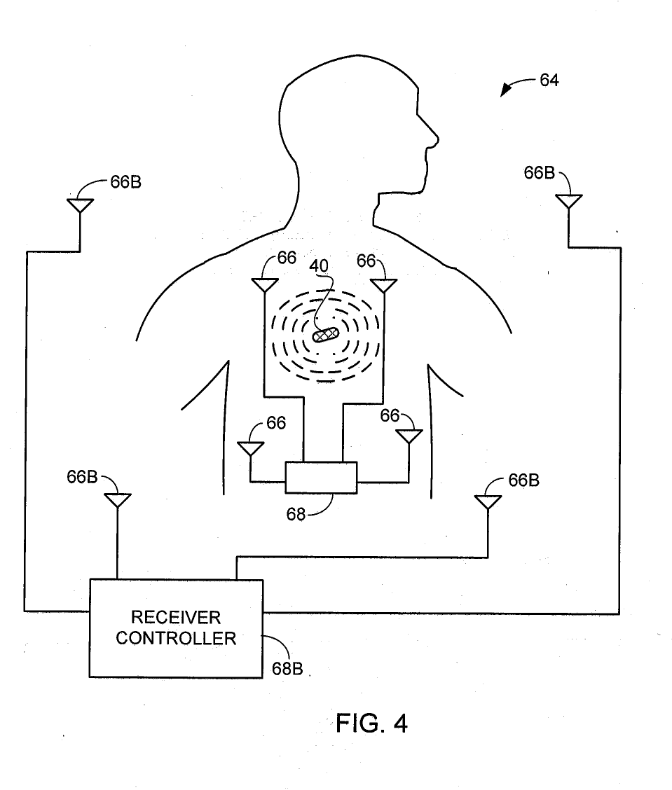

FIG. 4 schematically illustrates another embodiment of a diagnostic system 64 which can be used in applications where a diagnostic capsule 40 is ingested by a subject 58 and moves through their alimentary canal. The embodied diagnostic system 64 has at least three receivers 66 coupled to a receiver controller 68, all of which are located external to the subject 58. Since receivers 66 are configured to receive transmissions from diagnostic capsule 40, the receivers 66 should be positioned within transmission range of the diagnostic capsule 40. The receiver controller 68 may be configured to store the transmitted data received at the receivers 66 from the diagnostic capsule 40. The receivers 66, and optionally the receiver controller 68 may be portable and even wearable so that the subject 58 may carry the receivers 66 with him/her for increased freedom of movement during the relatively long time it can take a diagnostic capsule 40 to move through the alimentary canal. Receivers 66 and receiver controller 68 are illustrated in this embodiment as being worn by and/or attached to subject 58.

An alternate arrangement for the receivers and receiver controller is also illustrated in FIG. 4. The alternate receivers and receiver controller are labeled 66B and 68B, respectively. Alternate receivers 66B and alternate receiver controller 68B are not worn by or attached to subject 58. It should be understood that only one set of receivers/receiver controller (either 66 & 68 or 66B & 68B) is needed for the diagnostic system 64. For convenience, only the receivers 66 and receiver controller 68 will be discussed further.

The at least three receivers 66 are located in separate locations so that the receiver controller 68 may be configured to determine a position of the diagnostic capsule 40 relative to the at least three receivers 66 based on RF telemetry when the diagnostic capsule 40 is transmitting data. The receiver controller 68 may also be configured to store the determined location of the diagnostic capsule 40 relative to the at least three receivers 66 for one or more data transmissions received from the transmitter. Furthermore, if the location of the test subject 58 relative to the at least three receivers 66 is known by the receiver controller 68, then the receiver controller 68 may be configured to determine a location of the diagnostic capsule 40 within the subject 58 based on the RF telemetry determination and the relationship between the subject 58 being tested and the at least three receivers 66. Such a diagnostic system 64 has the advantage that positional information corresponding to the data being stored by the receiver controller 68 can be available in some embodiments to assist medical professionals in physically reaching target condition areas via follow-up surgery or similar procedures.

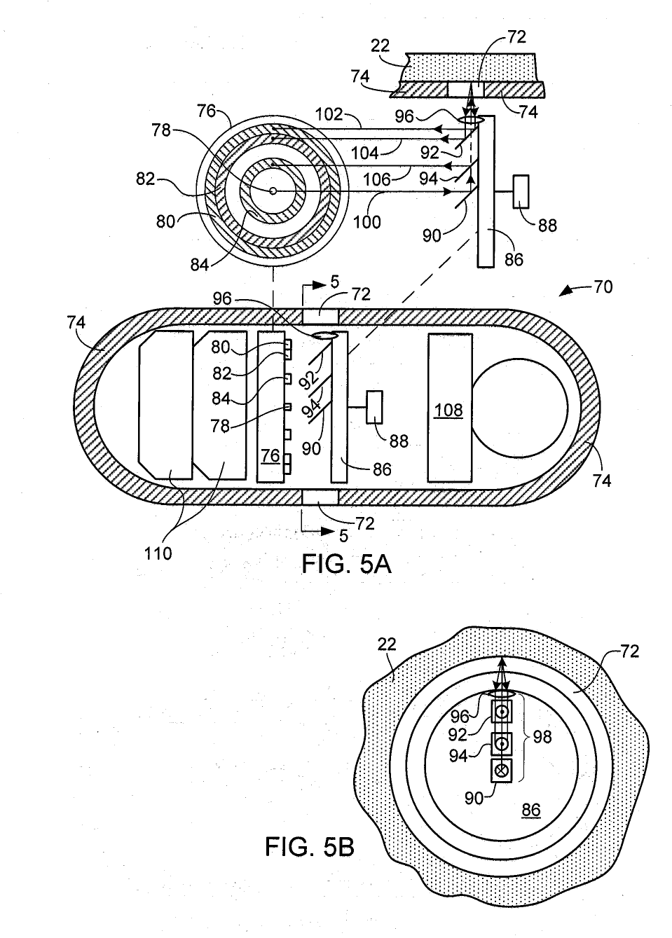

FIG. 5A schematically illustrates a cross-sectional view of another embodiment of a diagnostic capsule 70 with selected details highlighted and rotated. The diagnostic capsule 70 has a circumferential window 72 which is coupled to the capsule's casing 74. In other embodiments, the window 72 can be smaller or larger. A detector substrate 76 supports or is integral with a light source 78, a first annular detector region 80, a second annular detector region 82, and an annular imaging region 84. The annular detector regions 80, 82 may be made spectrally selective by similarly shaped optical filtering films either deposited directly or attached to the top of the detector regions. The light source 78 may be, for example, one or more LED dies, one or more laser dies, or any combination thereof. The one or more wavelengths of the light source 78 may be selected for illumination purposes (for example when interested in imaging) and/or for excitation purposes (for example when looking at spectroscopic signatures for various types of tissue, such as precancerous and cancerous tissues). For example, please see “Non-invasive Fluorescence-based Instrumentation for Cancer and Precancer Detection and Screening” by A. Katz and R. R. Alfano from Vol. 3913 of the Proceedings of SPIE, which is hereby incorporated by reference. Ratios of fluoresced light of differing wavelengths, for example 340 nm and 440 nm can provide valuable information for tissue classification and identification. Other techniques for evaluating reflected, backscattered, fluoresced, and/or luminesced light may also be used, and are encompassed within the scope of this patent application. Furthermore, any such analyses do not necessarily have to be limited to specific wavelengths, depending on the embodiment.

In this embodiment, however, the first annular detector region 80 is configured to sense light at a first wavelength, while the second annular detector region 82 is configured to sense light at a second wavelength. The nature of the annular regions can be seen more clearly in the rotated and highlighted depiction of the detector substrate 76 which is shown above the diagnostic capsule 70. The annular imaging region 84 is configured to sense one or more visible wavelengths of light.

The diagnostic capsule 70 also has a rotatable substrate 86 having a rotation axis which is substantially parallel to a window axis of the circumferential window 72. A motor 88 is rotatably coupled to the rotatable substrate 86. The rotatable substrate 86 has a mirror 90, a first beam splitter 92, a second beam splitter 94, and lens 96 which together make up an optical bench 98. (The optical bench is labeled in FIG. 5B) Returning to FIG. 5A, the highlighted and partially rotated portion of the drawing above the capsule 70 schematically illustrates the light path within the diagnostic capsule. Source light 100, which can be illumination light, excitation light, or both, is reflected by the mirror 90 through the beam splitters 94 and 92 and is focused by lens 96 through the window 72 and onto alimentary canal tissue 22. The size of the focused spot may vary, depending on the optical elements used and the how close the tissue 22 is to the window 72. The tissue reflects, scatters, fluoresces, and/or luminesces light back through the window 72. The first beam splitter 92 directs returning light of the first wavelength 102 and the second wavelength 104 to the first and second annular detector regions 80, 82. The second beam splitter 94 directs the remaining returning light 106 to the annular imaging region 84. The diagnostic capsule 70 may have circuitry (not shown) coupled to a transmitter 108 to transmit data corresponding to the light received by the detector regions 80, 82 and the imaging region 84. The diagnostic capsule 70 may also have a power source, such as one or more batteries 110.

FIG. 5B schematically illustrates a cross-sectional view of the diagnostic capsule of FIG. 5A taken along sectional lines 5-5 while the diagnostic capsule is within an alimentary canal.

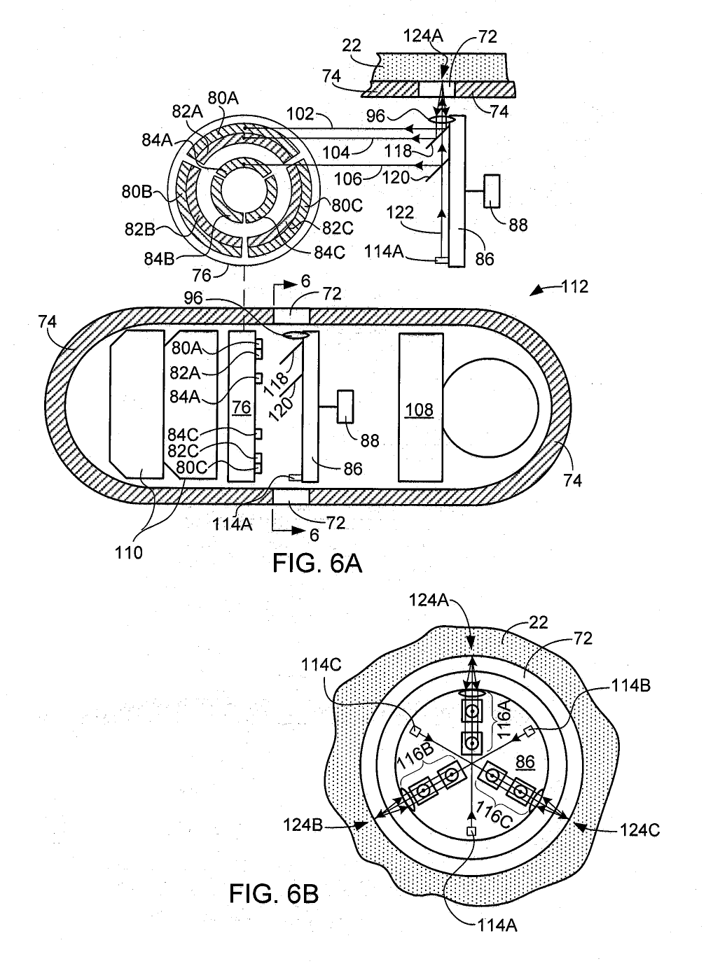

FIG. 6A schematically illustrates a cross-sectional view of another embodiment of a diagnostic capsule 112 with selected details highlighted and rotated. Diagnostic capsule 112 shares several components with the capsule embodiment 70 of FIG. 5A, therefore, in FIG. 6A, the similar components will be numbered with the same reference numerals and only a discussion of the different elements will be undertaken.

The detector substrate 76 has three separate groups (denoted with suffixes A, B, and C) of photo-sensitive regions. Group A has a first partially annular detector region 80A configured to sense light at a first wavelength, a second partially annular detector region 82A configured to sense light at a second wavelength, and a partially annular imaging region 84A configured to sense one or more wavelengths of light. Similarly, group B has a first partially annular detector region 80B configured to sense light at the first wavelength, a second partially annular detector region 82B configured to sense light at the second wavelength, and a partially annular imaging region 84B configured to sense one or more wavelengths of light. Similarly, group C has a first partially annular detector region 80C configured to sense light at the first wavelength, a second partially annular detector region 82C configured to sense light at the second wavelength, and a partially annular imaging region 84C configured to sense one or more wavelengths of light. The groups of photo-sensitive regions A, B, and C are isolated from each other. Three separate light sources 114A, 114B, and 114C and three separate optical benches 116A, 116B, and 116C are coupled to the rotatable substrate 86. The optical benches 116A, 116B, and 116C are preferably spaced around the rotatable substrate such that each one of the optical benches 116A, 116B, 116C will be aligned over just one of the photo-sensitive regions A, B, C as the rotatable substrate 86 turns, except during transitions from one photo-sensitive region to another. Light source 114A is positioned to provide source light to optical bench 116A, light source 114B is positioned to provide source light to optical bench 116B, and light source 114C is positioned to provide source light to optical bench 116C, regardless of which photo-sensitive region (A, B, or C) each optical bench is currently aligned with due to the rotation of the rotating substrate 86.

Each optical bench has a first beam splitter 118, a second beam splitter 120, and a lens 96. Source light 122 emitted by each light source 114, which can be illumination light, excitation light, or both, passes through the respective beam splitters 120 and 118 and is focused by each lens 96 through the window 72 and onto three simultaneous places 124A, 124B, and 124C on the alimentary canal tissue 22. (These three locations 124A, 124B, and 124C are better viewed in FIG. 6B). The size of the focused spot may vary, depending on the optical elements used and the how close the tissue 22 is to the window 72. The tissue reflects, scatters, fluoresces, and/or luminesces light back through the window 72 from each of the locations 124A, 124B, and 124C. Each of the first beam splitters 118 directs returning light of the first wavelength 102 and the second wavelength 104 to the first and second annular detector regions 80, 82 for each photo-sensitive region A, B, C. Each of the second beam splitters 120 directs the remaining returning light 106 to the annular imaging regions 84 for each photo-sensitive region A, B, C. The diagnostic capsule 112 may have circuitry (not shown) coupled to a transmitter 108 to transmit data corresponding to the light received by the detector regions 80A, 80B, 80C, 82A, 82B, 82C and the imaging regions 84A, 84B, and 84C. Although this embodiment features three detector regions, other similar embodiments may use any number of detector regions. This multiplicity of source/spots combined with the division of the sampled circumference into segments permits greater circumferential coverage per time or equivalently a slower rotation rate of the optical bench substrate for the same exposure as would be obtained for the single source/spot embodiment depicted in 5A]

FIG. 6B schematically illustrates a cross-sectional view of the diagnostic capsule of FIG. 6A taken along sectional lines 6-6 while the diagnostic capsule is within an alimentary canal.

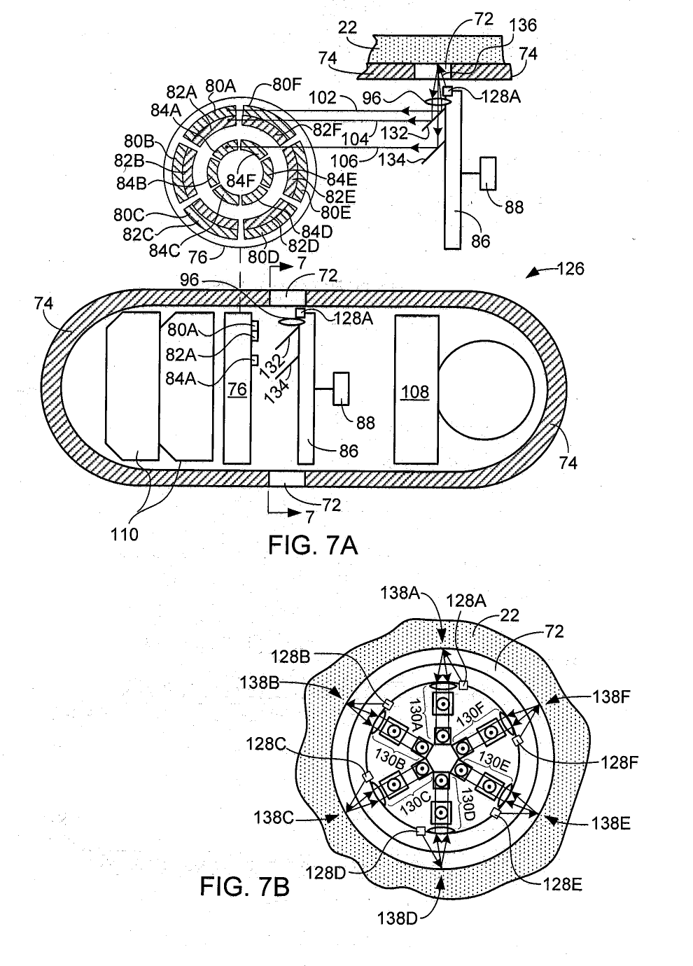

FIG. 7A schematically illustrates a cross-sectional view of another embodiment of a diagnostic capsule 126 with selected details highlighted and rotated. Diagnostic capsule 126 shares several components with the capsule embodiment 70 of FIG. 5A, therefore, in FIG. 7A, the similar components will be numbered with the same reference numerals and only a discussion of the different elements will be undertaken.

The detector substrate 76 has six separate groups (denoted with suffixes A, B, C, D, E, and F) of photo-sensitive regions. For simplicity, only group A is illustrated within the capsule 126, however, the multiple groups may be seen in the rotated view of detector substrate 76 above the capsule in FIG. 7A, as well as in FIG. 7B. Group A has a first partially annular detector region 80A configured to sense light at a first wavelength, a second partially annular detector region 82A configured to sense light at a second wavelength, and a partially annular imaging region 84A configured to sense one or more wavelengths of light. Similarly, group B has a first partially annular detector region 80B configured to sense light at the first wavelength, a second partially annular detector region 82B configured to sense light at the second wavelength, and a partially annular imaging region 84B configured to sense one or more wavelengths of light. Similarly, group C has a first partially annular detector region 80C configured to sense light at the first wavelength, a second partially annular detector region 82C configured to sense light at the second wavelength, and a partially annular imaging region 84C configured to sense one or more wavelengths of light. Similarly, group D has a first partially annular detector region 80D configured to sense light at the first wavelength, a second partially annular detector region 82D configured to sense light at the second wavelength, and a partially annular imaging region 84D configured to sense one or more wavelengths of light. Similarly, group E has a first partially annular detector region 80E configured to sense light at the first wavelength, a second partially annular detector region 82E configured to sense light at the second wavelength, and a partially annular imaging region 84E configured to sense one or more wavelengths of light. Similarly, group F has a first partially annular detector region 80F configured to sense light at the first wavelength, a second partially annular detector region 82F configured to sense light at the second wavelength, and a partially annular imaging region 84F configured to sense one or more wavelengths of light. The groups of photo-sensitive regions A, B, C, D, E, and F are isolated from each other.

Six separate light sources 128A, 128B, 128C, 128D, 128E, and 128F and six separate optical benches 130A, 130B, 130C, 130D, 130E, and 130F are coupled to the rotatable substrate 86. The optical benches 130A-F are preferably spaced around the rotatable substrate 86 such that each one of the optical benches 130A-F will be aligned over just one of the photo-sensitive regions A-F as the rotatable substrate 86 turns, except during transitions from one photo-sensitive region to another. Light sources 128A-F are positioned to provide source light to optical benches 130A-F respectively, regardless of which photo-sensitive region A-F each optical bench is currently aligned with due to the rotation of the rotating substrate 86. As opposed to the embodiment of FIG. 6A, the light sources in the embodiment of FIG. 7A are off-axis from the fluorescence-collecting optical benches and may have independent focusing optics.

Each optical bench has a first beam splitter 132, a mirror 134, and a lens 96. Source light 136 emitted by each light source 128, which can be illumination light, excitation light, or both, passes through the window 72 and onto six simultaneous places 138A-138F on the alimentary canal tissue 22. (These six locations 138A-F are better viewed in FIG. 7B). The size of the focused spot may vary, depending on the optical elements used and the how close the tissue 22 is to the window 72. The tissue reflects, scatters, fluoresces, and/or luminesces light back through the window 72 from each of the locations 138A-F. Each of the first beam splitters 132 directs returning light of the first wavelength 102 and the second wavelength 104 to the first and second annular detector regions 80, 82 for each photo-sensitive region A-F. Each of the mirrors 134 directs the remaining returning light 106 to the annular imaging regions 84 for each photo-sensitive region A-F. The diagnostic capsule 126 may have circuitry (not shown) coupled to a transmitter 108 to transmit data corresponding to the light received by the detector regions 80A-F, 82A-F and the imaging regions 84A-F. Although this embodiment features six detector regions, other similar embodiments may use any number of detector regions.

While each of the diagnostic capsule embodiments illustrated in FIGS. 6 and 7 are shown having separate optical benches for each of the illumination and light collection regions, it is envisioned that these separate optical systems may be comprised of arrays of optical components that are mechanically coupled. For example, the lens systems may comprise one or more arrays of lenses. The use of such arrays may enable more cost effective manufacturing than the use of separate microoptic components assembled into each of the benches. The optical benches and light sensitive regions may also be integrated into one or more substrates, again taking advantage of cost-effective manufacturing processes. Such processes may include those common to MEMS foundries and include Si wafer processing, deposition of one or more layers of optically transparent materials, release of optical elements, and pick and place assembly of additional components.

FIG. 7B schematically illustrates a cross-sectional view of the diagnostic capsule of FIG. 7A taken along sectional lines 7-7 while the diagnostic capsule is within an alimentary canal.

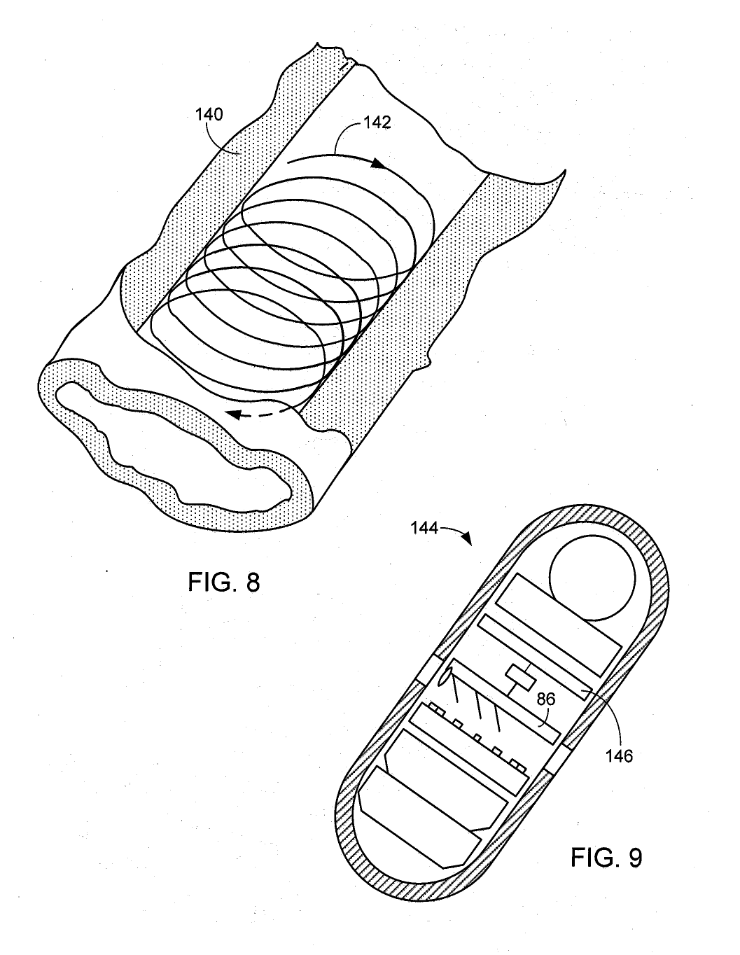

One of the advantages provided by the embodiments illustrated in FIGS. 6A, 6B, 7A, and 7B is that multiple sets of peripheral image data are being gathered for each 360 degree rotation of the rotatable substrate 86. By contrast, FIG. 8 is a partially exposed view of a portion of an alimentary canal 140 schematically illustrating a spot scanning path 142 from a diagnostic capsule as it passes through the alimentary canal 140. The diagnostic capsule (not shown) will be moving at some speed through the canal 140, and the rotation substrate will be rotating at another speed. This will result in a spiral-like scanning path 142 for each optical bench. In the illustrated embodiment, there is only one path 142, so the diagnostic capsule producing the path 142 only has one optical bench and associated at least one photo-sensitive region. It can be appreciated that the spiral scanning path 142 of the diagnostic capsule can leave undesirable gaps in data if the rotation rate of the rotating substrate is too slow compared to the speed the capsule is traveling through the canal 140. To compensate for this, higher rotation speeds may be used by the diagnostic capsule, either based on a pre-programmed value or based on a measurement of the traveling speed of the capsule. Another way to reduce the number of data gaps is to use a diagnostic capsule with a plurality of optical bench/photo-sensitive region pairs. Each pair of optical bench and photo-sensitive region can provide a separate data scanning path 142 offset from each other to assist in filling in the gaps. Of course, even in embodiments having multiple data scanning paths 142, increasing the rotational speed of the rotatable substrate can also help to minimize gaps in data.

As the rotational speed of the rotatable substrate in the diagnostic capsule increases, so will the torque exhibited on the diagnostic capsule increase. The torque will tend to cause the whole capsule to twist which can throw-off measurements and/or increase patient discomfort. Therefore, it may be desirable in some embodiments to reduce or offset this torque. FIG. 9 schematically illustrates an embodiment of a diagnostic capsule 144 having an embodiment of a momentum compensator 146. The momentum compensator 146 can be rotated in a direction opposite to the rotatable substrate 86 to balance the torque on the capsule.

As the ingested diagnostic capsule moves through the alimentary canal, a record of fluoresced, back-scattered, and/or reflected light signals may be made along with a record of the capsule's corresponding position. This record can then be transformed into an endoscopic diagnostic map to assist medical professionals with identifying and locating abnormal tissue.

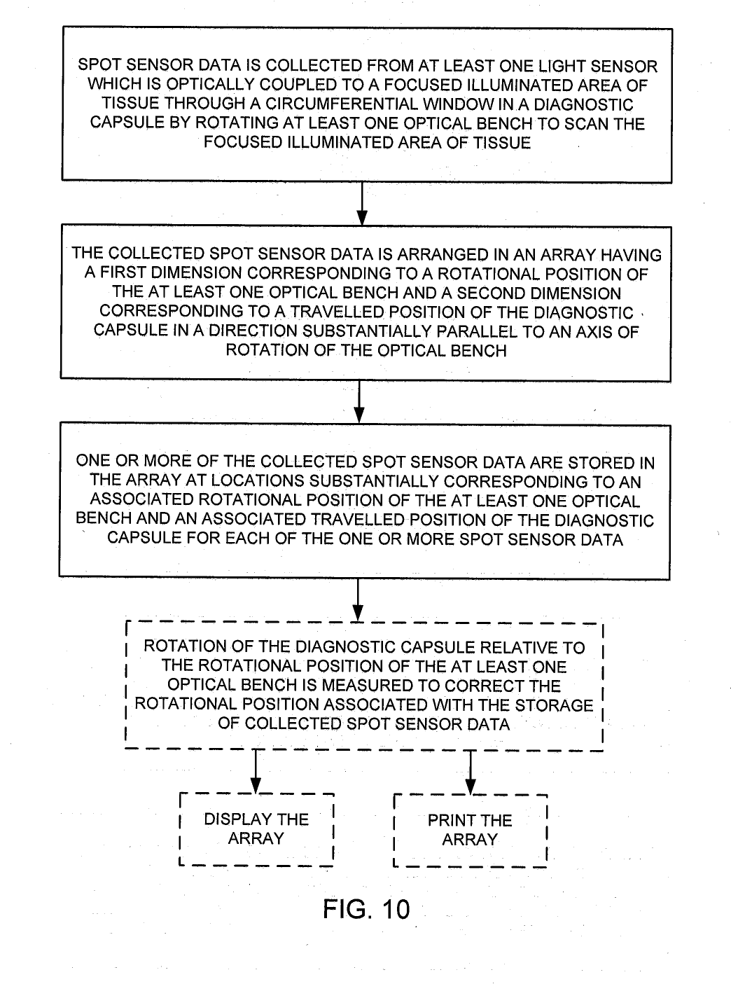

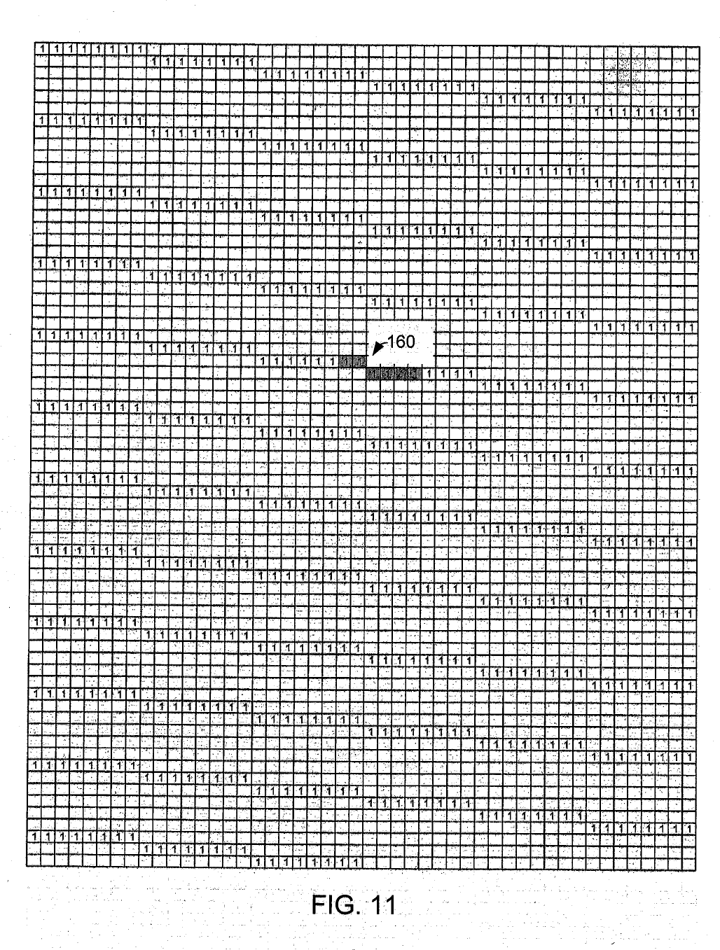

For example, FIG. 10 illustrates an embodiment of a method of generating an endoscopic diagnostic map. Spot sensor data are collected 148 from at least one light sensor which is optically coupled to a focused illuminated area of tissue through a circumferential window in a diagnostic capsule by rotating at least one optical bench to scan the focused illuminated area of tissue. As discussed above, more data paths can be collected if using more than one optical bench. The collected spot sensor data are arranged 150 in an array having a first dimension corresponding to a rotational position of the at least one optical bench and a second dimension corresponding to a traveled position of the diagnostic capsule in a direction substantially parallel to an axis of rotation of the optical bench. The traveled position of the diagnostic capsule may be determined in some embodiments by assuming a rate of travel and multiplying the assumed rate of travel by a travel time. In other embodiments, the traveled position of the diagnostic capsule may be determined by measuring a rate of travel of the diagnostic capsule and multiplying the measured rate of travel by the travel time. The rate of travel can be measured on-board the diagnostic capsule, for example with the help of an accelerometer. Alternatively, the rate of travel may be measured internally or externally if positional information is being tracked by the capsule. One or more of the collected spot sensor data are stored 152 in the array at locations substantially corresponding to an associated rotational position of the at least one optical bench and an associated traveled position of the diagnostic capsule for each of the one or more spot sensor data. Optionally, the rotation of the diagnostic capsule relative to the rotational position of the at least one optical bench can be measured 154 to correct the rotational position associated with the storage of collected spot sensor data. Optionally, the array corresponding to the endoscopic diagnostic map may be displayed 156 or printed 158.

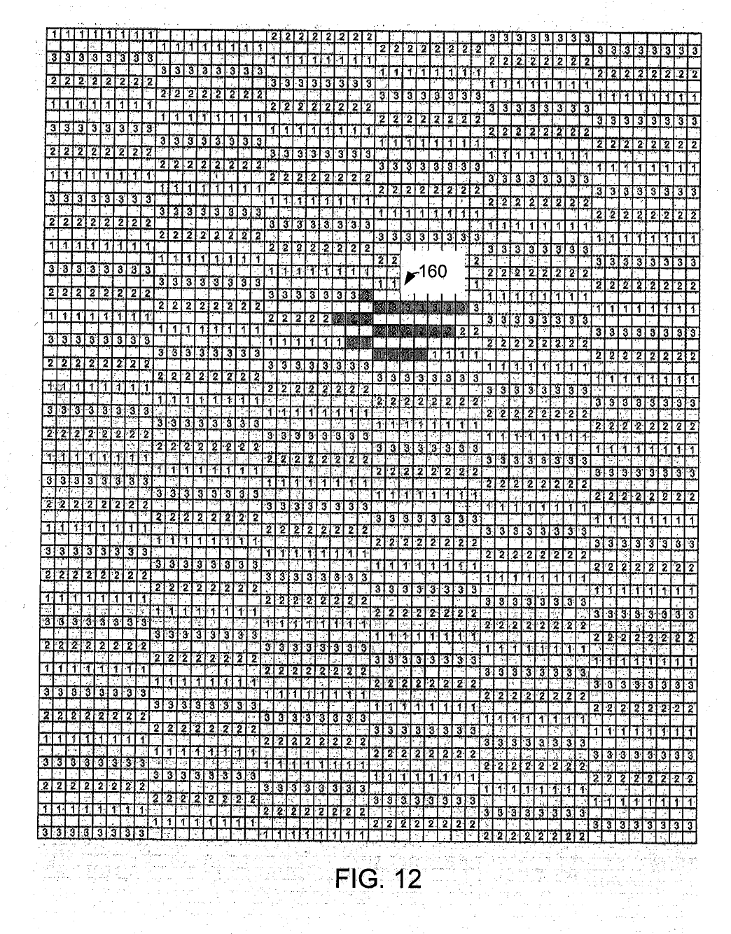

FIGS. 11 and 12 schematically illustrate embodiments of endoscopic diagnostic maps. The endoscopic diagnostic map embodied in FIG. 11 is based on a single optical bench embodiment. The horizontal dimension corresponds to rotational position, and the vertical direction corresponds to a traveled position. A number “1” is indicated in the map to show where data points were actually taken. A cluster of excitation-based data indicating an area of interest 160 is visible in this example. In other embodiments, straight image data may be displayed. In still further embodiments, ratios of different excitation-based data sets may be displayed. The endoscopic diagnostic map embodied in FIG. 12 is based on a triple optical bench embodiment. The horizontal dimension still corresponds to rotational position, and the vertical direction still corresponds to a traveled position. The numbers “1”, “2”, and “3” are indicated in the map to show where data points were actually taken and from which optical bench. The illustrated data in FIG. 12 correspond in position to the example from FIG. 11. In the case of FIG. 12, however, since there are more data available, a better picture of the area of interest 160 can be seen. Further embodiments do not need to show the numbers corresponding to the optical benches, but rather can simply show the image or spectral data.

Having thus described several embodiments of the claimed invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and the scope of the claimed invention. Additionally, the recited order of the processing elements or sequences, or the use of numbers, letters, or other designations therefore, is not intended to limit the claimed processes to any order except as may be specified in the claims. Accordingly, the claimed invention is limited only by the following claims and equivalents thereto.

What is claimed is:

1. A diagnostic capsule, comprising:
at least one light source;
a rotatable substrate having at least one optical bench;
at least one photo-sensitive region; and
a circumferential window having an axis substantially parallel to an axis of rotation of the rotatable substrate.

2. The diagnostic capsule of claim 1, wherein the at least one optical bench is further configured:
to focus outgoing light from the at least one light source through the circumferential window; and
to focus incoming light entering the circumferential window onto the at least one photosensitive region.

3. The diagnostic capsule of claim 1, further comprising a motor coupled to the rotatable substrate.

4. The diagnostic capsule of claim 3, further comprising a momentum compensator coupled to the motor.

5. The diagnostic capsule of claim 1, wherein the at least one light source is selected from the group consisting of an excitation light source, an illumination light source, a light emitting diode (LED), and a laser.

6. The diagnostic capsule of claim 1, wherein the at least one photo-sensitive region is selected from the group consisting of an ultraviolet (UV) detector and an imaging detector.

7. The diagnostic capsule of claim 1, wherein the circumferential window is selected from the group consisting of glass, quartz, optically transparent metal, plastic, and combinations thereof.

8. The diagnostic capsule of claim 1, wherein the circumferential window is fully transparent.

9. The diagnostic capsule of claim 1, further comprising circuitry operatively coupled to the at least one photo-sensitive region and configured to sample the at least one photo-sensitive region to capture spot light data at intervals from light directed to the at least one photo-sensitive region by the at least one optical bench.

10. The diagnostic capsule of claim 9, wherein the intervals are selected from the group consisting of varying regular intervals which change depending on an interval configuration, regular intervals, and irregular intervals.

11. The diagnostic capsule of claim 9, further comprising a transmitter coupled to the circuitry.

12. A diagnostic system, comprising:
a) a diagnostic capsule, comprising:
at least one light source;
a rotatable substrate having at least one optical bench;
at least one photo-sensitive region;
an at least partially transparent circumferential window having an axis substantially parallel to an axis of rotation of the rotatable substrate; and
a transmitter configured to transmit sensor data from the at least one photo-sensitive region;
b) at least one receiver configured to receive the sensor data from the transmitter; and
c) a receiver controller coupled to the at least one receiver, the receiver controller configured to store the sensor data.

13. The diagnostic system of claim 12, further comprising means for determining one or more locations of the diagnostic capsule as it moves through at least a portion of an alimentary canal.

14. The diagnostic system of claim 13, wherein the receiver controller is further configured to store the one or more determined locations of the diagnostic capsule such that the one or more determined locations are coupled with the sensor data.

15. The diagnostic system of claim 14, further configured to generate a diagnostic map of sensor data.

16. A method of generating an endoscopic diagnostic map, comprising:
collecting spot sensor data from at least one light sensor which is optically coupled to a focused illuminated area of tissue through a circumferential window in a diagnostic capsule by rotating at least one optical bench to scan the focused illuminated area of tissue;
arranging the collected spot sensor data in an array having a first dimension corresponding to a rotational position of the at least one optical bench and a second dimension corresponding to a traveled position of the diagnostic capsule in a direction substantially parallel to an axis of rotation of the optical bench; and
wherein one or more of the collected spot sensor data are stored in the array at locations substantially corresponding to an associated rotational position of the at least one optical bench and an associated traveled position of the diagnostic capsule for each of the one or more spot sensor data.

17. The method of claim 16, further comprising measuring rotation of the diagnostic capsule relative to the rotational position of the at least one optical bench to correct the rotational position associated with the storage of collected spot sensor data.

18. The method of claim 16, wherein the traveled position of the diagnostic capsule is determined by assuming a rate of travel and multiplying the assumed rate of travel by a travel time.

19. The method of claim 16, wherein the traveled position of the diagnostic capsule is determined by measuring a rate of travel and multiplying the measured rate of travel by a travel time.

20. The method of claim 16, wherein the collected spot sensor data is selected from the group consisting of ultraviolet (UV) data and image data.

21. The method of claim 16, further comprising displaying the array.

22. The method of claim 16, further comprising printing the array.

23. An endoscopic diagnostic map, comprising:
a first dimension corresponding to a rotational position of at least one optical bench on a rotatable substrate of a diagnostic capsule; and
a second dimension corresponding a traveled position of the diagnostic capsule.

24. The endoscopic diagnostic map of claim 23, further comprising collected endoscopic spot sensor data distributed within the diagnostic map at locations based on a rotational position of the at least one optical bench and a traveled position of the diagnostic capsule for each of the collected spot sensor data.

25. A diagnostic capsule, comprising:
a) an illumination light source;
b) an excitation light source;
c) a first annular detector region configured to sense light at a first wavelength;
d) a second annular detector region configured to sense light at a second wavelength;
e) an annular imaging region configured to sense one or more visible wavelengths of light;
f) a circumferential window having a window axis;
g) a rotatable substrate having a rotation axis which is substantially parallel to the window axis and comprising an optical bench configured to:
focus light from the illumination and excitation light sources on a spot outside the circumferential window; and
focus reflected, backscattered, luminesced, or fluoresced light of the first wavelength from outside the circumferential window onto the first annular detector region;
focus reflected, backscattered, luminesced, or fluoresced light of the second wavelength from outside the circumferential window onto the second annular detector region; and
focus reflected, backscattered, luminesced, or fluoresced light of the one or more visible wavelengths from outside the circumferential window onto the annular imaging region;
h) a motor rotatably coupled to the rotatable substrate; and
i) circuitry configured to transmit data based on the reflected, luminesced, or fluoresced light focused on the first annual detector region, the second annular detector region, and the annular imaging region.
